(19) United States
(12) Patent Application Publication
(10) Pub. No.: US 2019/0321375 A1
(43) Pub. Date: Oct. 24, 2019

(54) METHOD OF ADMINISTERING A NEUROSTEROID TO EFFECT ELECTROENCEPHALOGRAPHIC (EEG) BURST SUPPRESSION

(71) Applicant: MARINUS PHARMACEUTICALS, INC., Radnor, PA (US)

(72) Inventors: Michael SAPORITO, West Chester, PA (US); Albena PATRONEVA, Wayne, PA (US); David CZEKAI, Haverford, PA (US)

(21) Appl. No.: 16/341,598

(22) PCT Filed: Oct. 13, 2017
(86) PCT No.: PCT/US17/56565

§ 371 Date: Apr. 12, 2019

Related U.S. Application Data
(60) Provisional application No. 62/408,330, filed on Oct. 14, 2016.
(60) Provisional application No. 62/486,781, filed on Apr. 18, 2017.

(51) Int. Cl. A61K 31/57; A61K 47/32; A61K 47/36; A61K 9/00; A61P 25/08; A61K 9/51

(52) U.S. Cl. CPC A61K 31/57; A61K 47/32; A61K 47/36; A61K 9/5161; A61P 25/08; A61K 9/5138; A61K 9/0019

(57) ABSTRACT

The disclosure provides a method of eliciting electroencephalographic burst suppression or electroencephalographic suppression in a patient. the method includes administering to the patient a formulation comprising neurosteroid nanoparticles having a D50 of less than 2 microns and a polymeric surface stabilizer chosen from hydroxyethyl starch, dextran, and povidone and 0.1 to 50 mg of the neurosteroid per 1 kg of the patient's body weight The neurosteroid may be administered intravenously, intramuscularly, subcutaneously, or orally. Continuous intravenous administration and intravenously, intramuscularly, subcutaneously, or orally administering sequential bolus doses comprising 0.5 mg of ganaxolone per 1 kg of body weight in a human patient, with an interval of less than 30 minutes between two consecutive doses are included in the disclosure.

### CROSS REFERENCE TO RELATED APPLICATION

This application claims priority from U.S. Provisional Application No. 62/408,330, filed Oct. 14, 2016 and U.S. Provisional Application No. 62/486,781, filed Apr. 18, 2017, both of which are incorporated by reference in their entirety.

### BACKGROUND

Pregnane neurosteroids are a class of compounds useful as anesthetics, sedatives, hypnotics, and anticonvulsants. These compounds are marked by very low solubility, which limits their formulation options. Injectable formulations of pregnane neurosteroids are particularly desirable as these compounds are used for clinical indications for which oral administration is precluded, such as anesthesia and for the treatment of active seizures.

Status epilepticus (SE) is a serious seizure disorder in which the epileptic patient experiences a seizure lasting more than five minutes, or more than one seizure in a five minute period without recovering between seizures. In certain instances, convulsive seizures may last days or even weeks. Status epilepticus is treated in the emergency room with conventional anticonvulsants. GABAA receptor modulators such as benzodiazepines (BZs) are a first line treatment. Patients who fail to respond to BZs alone are usually treated with anesthetics or barbiturates in combination with BZs. About 23-43% of status epilepticus patients who are treated with a benzodiazepine and at least one additional antiepileptic drug fail to respond to treatment and are considered refractory (Rossetti, A. O. and Lowenstein, D. H., Lancet Neurol. (2011) 10 (10): 922-930.) There are currently no good treatment options for these patients. The mortality rate for refractory status epilepticus (RSE) patients is high and most RSE patients do not return to their pre-RSE clinical condition. About 15% of patients admitted to hospital with SE progress to RSE and even to super-refractory SE (SRSE), in which the patients have continued or recurrent seizures 24 hours or more. These patients are being treated with anesthetic therapy and periodically weaned off the general anesthetic to access their therapeutic response. SRSE is associated with high rates of mortality and morbidity. (Shorvon, S., and Ferlisi, M., Brain, (2011) 134 (10): 2802-2818.)

Another serious seizure disorder is PCDH19 female pediatric epilepsy, which affects approximately 1,500-3,000 females in the United States. This genetic disorder is associated with seizures beginning in the early years of life, mostly focal clustered seizures that can last for weeks. The mutation of the PCDH19 gene has been associated with low levels of allopregnanolone. The patients are often hospitalized during clusters and require IV therapy.

Neurosteroids, including ganaxolone, are known to have anesthetic properties, due to their role in modulating neuronal excitability. It is thought that neurosteroids inhibit nicotinic acetylcholine receptors, the target of general anesthetics.

Burst suppression is an EEG pattern characterized by periods of high-voltage electrical activity alternating with periods of no brain activity. The burst suppression pattern is present in persons with inactivated brain states, such as anesthetized or comatose patients.

There exists the need for additional treatments for seizure disorders that can be treated with agents that effect burst suppression. These seizure disorders include status epilepticus, refractory status epilepticus, super refractory status epilepticus, and PCDH19 female pediatric epilepsy. There also exists a need for additional sedative or anesthetic agents. This disclosure fulfills this need by providing injectable pregnane neurosteroid formulations and provides additional advantages that are described herein.

### SUMMARY

The disclosure provides a method of administering a neurosteroid formulation to elicit electroencephalographic (EEG) burst suppression/EEG suppression. The method includes administering to the patient a formulation comprising neurosteroid nanoparticles having a D50 of less than 2 microns and a polymeric surface stabilizer chosen from hydroxyethyl starch, dextran, and povidone and 0.1 to 250 mg of neurosteroid per 1 kg of the patient's body weight. The formulation can be administered as sequential bolus doses with an interval of less than 30 minutes between two consecutive doses. In certain embodiments the neurosteroid is a compound of Formula I (below) or a pharmaceutically acceptable salt of such a compound.

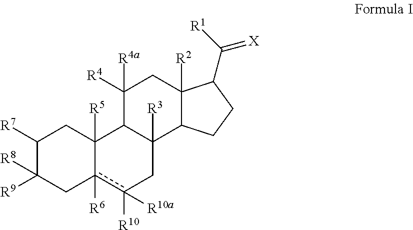

or a pharmaceutically acceptable salt thereof, wherein:

[symbol] is a double or single bond;

X is O, S, or NR¹¹;

R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;

R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆carbocycle;

R^{4a} is hydrogen or R⁴ and R^{4a} are taken together to form an oxo (═O) group;

R², R³, R⁵, and R⁶, are each independently hydrogen, hydroxyl, halogen, optionally substituted alkyl, optionally substituted cycloalkyl, or optionally substituted (cycloalkyl)alkyl, or optionally substituted heteroalkyl;

R⁷ is hydrogen, halogen, optionally substituted alkyl, optionally substituted C₃-C₆carbocycle, optionally substituted (C₃-C₆carbocycle)alkyl or —OR⁷⁰ where R⁷⁰ is hydrogen, optionally substituted alkyl, optionally substituted C₃-C₆carbocycle, or optionally substituted (C₃-C₆carbocycle)alkyl;

R⁸ is hydrogen, optionally substituted alkyl or optionally substituted C₃-C₆carbocycle, and R⁹ is hydroxyl; or

R⁸ and R⁹ are taken together to form an oxo group;

R¹⁰ is hydrogen, halogen, hydroxyl, optionally substituted alkyl, optionally substituted heteroalkyl, optionally substituted C₃-C₆carbocyle, or optionally substituted (C₃-C₆carbocycle)alkyl, and R^{10a} is hydrogen, halogen, or optionally substituted alkyl, provided that if [symbol] is a double bond R^{10a} is absent;

each alkyl is a C₁-C₁₀alkyl and optionally contains one or more single bonds replaced by a double or triple bond;

each heteroalkyl group is an alkyl group in which one or more methyl group is replaced by an independently chosen —O—, —S—, —N(R¹¹)—, —S(═O)— or —S(═O)₂—, where R¹¹ is independently chosen at each occurrence and is hydrogen, alkyl, or alkyl in which one or more methylene group is replaced by —O—, —S—, —NH, or —N-alkyl.

In certain embodiments the neurosteroid is ganaxolone. In other embodiments the neurosteroid is allopregnanolone, alphaxalone, minaxolone, allotetrahydrodeoxycorticosterone, etiocholanone, dehydroepiandrosterone (including dehydroepiandrosterone sulfate), or pregnanolone (including pregnanolone sulfate). Administration of the neurosteroid can be through intravenous, intramuscular, subcutaneous or oral administration. The administration can be a single (bolus) administration, repeat administration with intervals between 3 and 30 minutes apart or by continuous infusion via IV drip or intramuscular or subcutaneous depot.

The disclosure also provides a method for detecting efficacious dose-levels of ganaxolone for treatment of an epileptic condition or effecting anesthesia. According to the method, sequential bolus doses of a formulation comprising 0.1 to 50 mg of the neurosteroid per 1 kg of patient body weight are administered to the patient intravenously, intramuscularly, or subcutaneously with an interval of less than 30 minutes between two consecutive doses An electroencephalography pattern in a brain of the patient is then continuously measured. After administration of a cummulative efficacious dose, electroencephalographic burst suppression/EEG suppression in the electroencephalography pattern is detected. The amount of neurosteroid needed to produce the detected electroencephalographic burst suppression in the electroencephalography pattern is determined to be the efficacious dose. The neurosteroid plasma level produced by the efficacious dose is subsequently maintained in the patient to remedy the epileptic condition or effect anesthesia.

### BRIEF DESCRIPTION OF THE DRAWINGS

The above and other aspects and features of the present disclosure will become more apparent by describing in detail exemplary embodiments thereof with reference to the attached drawings, in which:

FIG. 1 shows EEG burst suppression/EEG suppression patterns obtained at various doses of the ganaxolone formulation;

FIG. 2A is a graph of the tail reflex score versus time from the first dose (minutes, min) illustrating the results of the tail pinch experiments in which the ganaxolone formulation was administered every three minutes; and

FIG. 2B is a graph of the tail reflex score versus time from the first dose (minutes, min) illustrating the results of the tail pinch experiments in which the ganaxolone formulation was administered every 30 minutes.

FIG. 3A. Mean plasma ganaxolone concentration (ng/mL) for 2 hours for ganaxolone hydroxyethyl starch formulation and positive control ganaxolone Captisol formulation in rats after a single intravenous injection, 12 mg/kg dose. FIG. 3B, Mean brain ganaxolone concentration (ng/mL) for 2 hours for ganaxolone hydroxyethyl starch formulation and positive control ganaxolone/Captisol formulation in rats after a single intravenous injection, for 12 mg/kg dose. Ganaxolone nanoparticle formulation provided substantially higher brain concentration of ganaxolone than the ganaxolone/Captisol solution formulation.

FIG. 4A. shows a dose response curve for ganaxolone nanosuspension formulations. EEG power was monitored in rats following administration of pilocarpine to induce seizure (Time—15 minutes), followed by IV administration of a ganaxolone bolus at Time 0.

FIG. 4B. shows a comparison of the effects of a ganaxolone nanosuspension formulation and a fully solubilized CAPTISOL formulation on EEG power in the pilocarpine induced seizure model.

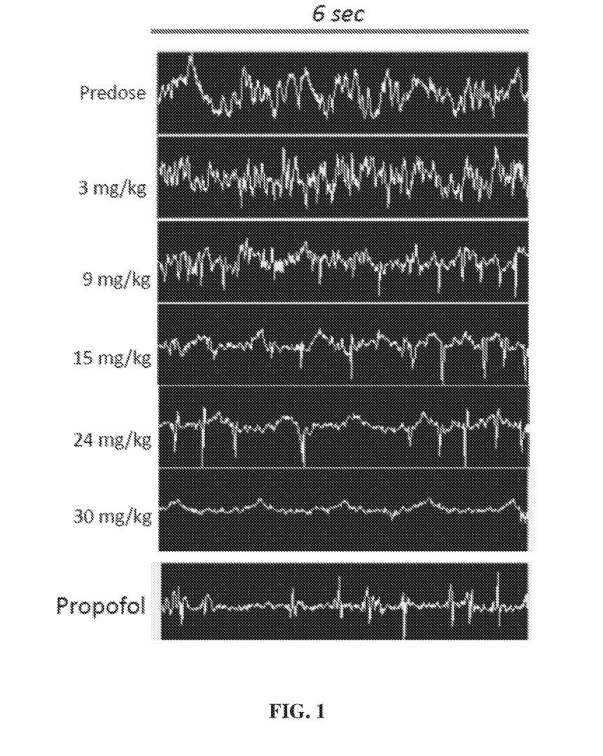

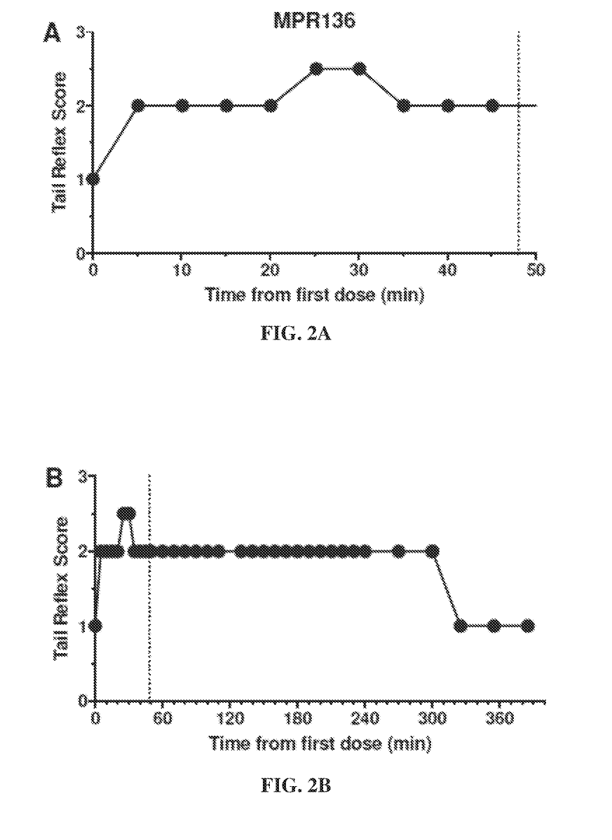

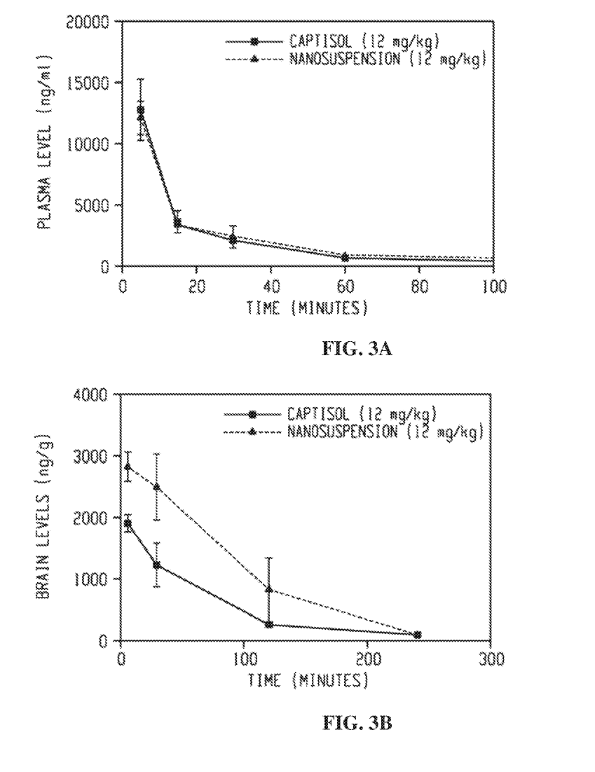

### DETAILED DESCRIPTION

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed. No language in the specification should be construed as indicating any non-claimed element as essential to the practice of the invention.

The terms “a” and “an” do not denote a limitation of quantity, but rather denote the presence of at least one of the referenced item.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe. Thus compositions slightly outside the cited ranges are also encompassed by the scope of the present claims.

The terms “comprising,” “including,” and “containing” are non-limiting. Other non-recited elements may be present in embodiments claimed by these transitional phrases. Where “comprising,” “containing,” or “including” are used as transitional phrases other elements may be included and still form an embodiment within the scope of the claim. The open-ended transitional phrase “comprising” encompasses the intermediate transitional phrase “consisting essentially of” and the close-ended phrase “consisting of.”

A “bolus dose” is a relatively large dose of medication administered in a short period, for example, within 1 to 30 minutes.

“Infusion” administration is a non-oral administration, typically intravenous though other non-oral routes such as epidural administration are included in some embodiments. Infusion administration occurs over a longer period than a bolus administration, for example, over a period of at least 15 minutes, at least 30 minutes, at least 1 hour, at least 2 hours, at least 3 hours, or at least 4 hours.

“Alkyl” is a branched or straight chain saturated aliphatic hydrocarbon group, having the specified number of carbon atoms, generally from 1 to 8 carbon atoms. The term C₁-C₆-alkyl as used herein indicates an alkyl group having from 1, 2, 3, 4, 5, or 6 carbon atoms. Other embodiments include alkyl groups having from 1 to 6 carbon atoms, 1 to 4 carbon atoms or 1 or 2 carbon atoms, e.g. C₁-C₈-alkyl, C₁-C₄-alkyl, and C₁-C₂-alkyl. Examples of alkyl include, but are not limited to, methyl, ethyl, n-propyl, isopropyl, n-butyl, 3-methylbutyl, t-butyl, n-pentyl, and sec-pentyl. In this disclosure “alkyl” includes alkyl groups as described in which one or more C—C saturated bonds is replace by a double or triple bonds, i.e. alkenyl or alkynyl groups.

“Aryl” indicates aromatic groups containing only carbon in the aromatic ring or rings. Typical aryl groups contain 1 to 3 separate, fused, or pendant rings and from 6 to 18 ring atoms, without heteroatoms as ring members. When indicated, such aryl groups may be further substituted with carbon or non-carbon atoms or groups. Aryl groups include, for example, phenyl, naphthyl, including 1-naphthyl, 2-naphthyl, and bi-phenyl. An “arylalkyl” substituent group is an aryl group as defined herein, attached to the group it substitutes via an alkylene linker. The alkylene is an alkyl group as described herein except that it is bivalent.

“Carbocycle” is a saturated, unsaturated or aromatic cyclic group having the indicated number of ring atoms, with all ring atoms being carbon. “(Carbocycle)alkyl” is a carbocycle, as defined, attached to the group it substitutes via an alkyl linker.

“Cycloalkyl” is a saturated hydrocarbon ring group, having the specified number of carbon atoms. Monocyclic cycloalkyl groups typically have from 3 to 6 (3, 4, 5, or 6) carbon ring atoms. Cycloalkyl substituents may be pendant from a substituted nitrogen, oxygen, or carbon atom, or a substituted carbon atom that may have two substituents may have a cycloalkyl group, which is attached as a spiro group. Examples of cycloalkyl groups include cyclopropyl, cyclobutyl, cyclopentyl, and cyclohexyl. “(Cycloalkyl)alkyl” is a cycloalkyl group as described attached to the group it substitutes via an alkyl group, such as a C₁-C₄alkyl or C₁-C₂alkyl.

“Cₘₐₓ” is the measured concentration of an active concentration in the plasma at the point of maximum concentration.

A “heteroalkyl” group is an alkyl group as described with at least one carbon replaced by a heteroatom, e.g. N, O, or S.

A “heteroaryl” group is a stable monocyclic aromatic ring having the indicated number of ring atoms which contains from 1 to 4, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 4, or in some embodiments from 1 to 2, heteroatoms chosen from N, O, and S, with remaining ring atoms being carbon. Monocyclic heteroaryl groups typically have from 5 to 7 ring atoms. In certain embodiments there heteroaryl group is a 5- or 6-membered monocyclic heteroaryl group having 1, 2, 3, or 4 heteroatoms chosen from N, O, and S, with no more than 2 O atoms and 1 S atom. Examples of heteroaryl groups include thienyl, furanyl, oxazolyl, thiazolyl, imidazolyl, pyrrolyl, pyrazolyl, pyridyl, pyrimidinyl, and pyridizinyl groups.

A “patient” is a human or non-human animal in need of medical treatment. Medical treatment includes treatment of an existing condition, such as a disorder or injury. In certain embodiments treatment also includes prophylactic or preventative treatment, or diagnostic treatment.

“Pharmaceutical compositions” are compositions comprising at least one active agent, such as a compound or salt, solvate, or hydrate of Formula (I), and at least one other substance, such as a carrier. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as a seizure disorder.

“Povidone” also known as polyvidone and polyvinylpyrrolidone (PVP) is a water soluble polymer made from the monomer, N-vinylpyrrolidone. Plasdone C-12 and C-17 are pharmaceutical grade homopolymers of N-vinylpyrrolidone. Plasdone C-12 has a K value of 10-2-13.8 and nominal molecular weight of 4000 d. Plasdone C-17 has a K-value of 15.5-17.5 and nominal molecular weight of 10,000 d.

The term “substituted” as used herein, means that any one or more hydrogens on the designated atom or group is replaced with a selection from the indicated group, provided that the designated atom's normal valence is not exceeded. When the substituent is oxo (i.e., ═O) then 2 hydrogens on the atom are replaced. When an oxo group substitutes a heteroaromatic moiety, the resulting molecule can sometimes adopt tautomeric forms. For example a pyridyl group substituted by oxo at the 2- or 4-position can sometimes be written as a pyridine or hydroxypyridine. Combinations of substituents and/or variables are permissible only if such combinations result in stable compounds or useful synthetic intermediates. A stable compound or stable structure is meant to imply a compound that is sufficiently robust to survive isolation from a reaction mixture and subsequent formulation into an effective therapeutic agent. Unless otherwise specified, substituents are named into the core structure. For example, it is to be understood that aminoalkyl means the point of attachment of this substituent to the core structure is in the alkyl portion and alkylamino means the point of attachment is a bond to the nitrogen of the amino group.

Suitable groups that may be present on a “substituted” or “optionally substituted” position include, but are not limited to, e.g., halogen; cyano; —OH; oxo; —NH₂; nitro; azido; alkanoyl (such as a C₂-C₆ alkanoyl group); C(O)NH₂; alkyl groups (including cycloalkyl and (cycloalkyl)alkyl groups) having 1 to 8 carbon atoms, or 1 to 6 carbon atoms; alkenyl and alkynyl groups including groups having one or more unsaturated linkages and from 2 to 8, or 2 to 6 carbon atoms; alkoxy groups having one or more oxygen linkages and from 1 to 8, or from 1 to 6 carbon atoms; aryloxy such as phenoxy; alkylthio groups including those having one or more thioether linkages and from 1 to 8 carbon atoms, or from 1 to 6 carbon atoms; alkylsulfinyl groups including those having one or more sulfinyl linkages and from 1 to 8 carbon atoms, or from 1 to 6 carbon atoms; alkylsulfonyl groups including those having one or more sulfonyl linkages and from 1 to 8 carbon atoms, or from 1 to 6 carbon atoms; aminoalkyl groups including groups having one or more N atoms and from 1 to 8, or from 1 to 6 carbon atoms; mono- or dialkylamino groups including groups having alkyl groups from 1 to 6 carbon atoms; mono- or dialkylaminocarbonyl groups (i.e. alkylNHCO— or (alkyl₁)(alkyl₂)NCO—) having alkyl groups from 1 to 6 carbon atoms; aryl having 6 or more carbons. In certain embodiments substituents that may be present at an optionally substituted position include halogen, hydroxyl, —CN, —SH, nitro, oxo, amino, C₁-C₆alkyl, C₁-C₆alkoxy, C₁-C₆alkylthio, C₁-C₆alkylsulfonyl, mono- and di-(C₁-C₄alkyl)amino, mono- and di-C₁-C₄alkylcarboxamide, (C₃-C₆cyclalkyl)C₀-C₂alkyl, C₁-C₂haloalkyl; and C₁-C₂haloalkoxy.

A “therapeutically effective amount” or “effective amount” is that amount of a pharmaceutical agent to achieve a pharmacological effect. The term “therapeutically effective amount” includes, for example, a prophylactically effective amount. An “effective amount” of neurosteroid is an amount needed to achieve a desired pharmacologic effect or therapeutic improvement without undue adverse side effects. The effective amount of neurosteroid will be selected by those skilled in the art depending on the particular patient and the disease. It is understood that “an effective amount” or “a therapeutically effective amount” can vary from subject to subject, due to variation in metabolism of neurosteroid, age, weight, general condition of the subject, the condition being treated, the severity of the condition being treated, and the judgment of the prescribing physician.

“Treat” or “treatment” refers to any treatment of a disorder, such as inhibiting the disorder, e.g., arresting the development of the disorder, relieving the disorder, causing regression of the disorder or disease, relieving a condition caused by the disease or disorder, or reducing the symptoms of the disease or disorder. In the context of this disclosure treatment includes effecting anesthesia, arresting active seizures, and reducing the frequency and severity of seizure in a patient having a seizure disorder.

Neurosteriod “Nanoparticle suspension” or “nanoparticle dispersion” or “nanodispersion” refers to neurosteriod particles with a volume weighted median diameter (D50) of less than 2000 nm suspended in an aqueous medium and are used interchangeably.

### Chemical Description

The disclosure provides a method of administering a neurosteroid formulation to elicit electroencephalographic (EEG) burst suppression/EEG suppression.

The neurosteroid can be a compound of Formula I or salt thereof as discussed in the SUMMARY section. In certain embodiments the neurosteroid is allopregnanolone, ganaxolone, alphaxalone, alphadolone, hydroxydione, minaxolone, pregnanolone, acebrochol. The neurosteroid can be ganaxolone. The neurosteroid may also be a compound of Formula I having the formula

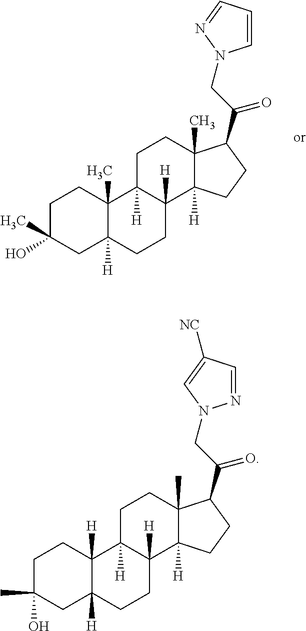

The neurosteroid can be administered intravenously, intramuscularly, subcutaneously or orally.

In certain embodiments the neurosteroid is administered sequential bolus doses, such as intravenous bolus doses, comprising 0.1 mg to 100 mg, 0.1 mg to 50 mg, 0.1 mg to 10 mg, at least 10 mg, at least 5 mg, at least 3 mg, at least 1 mg, at least 0.5 mg, at least 0.1 mg of neurosteroid per 1 kg of body weight with an interval of less than 30 minutes, less than 10 minutes, less than 5 minutes or 3 minutes between two consecutive doses. In certain embodiments at least three, more that 5 or more than 10 neurosteroid bolus doses are administered.

In certain embodiments the neurosteroid is ganaxolone. Ganaxolone (CAS Reg. No. 38398-32-2, 3α-hydroxy, 3β-methyl-5α-pregnan-20-one) is a synthetic steroid with anti-convulsant activity useful in treating epilepsy and other central nervous system disorders.

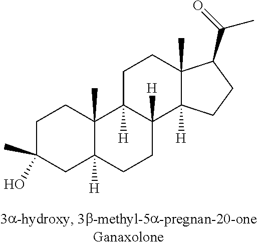

Ganaxolone has a relatively long half-life—approximately 20 hours in human plasma following oral administration (Nohria, V. and Giller, E., Neurotherapeutics, (2007) 4 (1): 102-105). Furthermore, ganaxolone has a short Tₘₐₓ, which means that therapeutic blood levels are reached quickly. Thus initial bolus doses (loading doses) may not be required, which represents an advantage over other treatments. Ganaxolone is useful for treating seizures in adult and pediatric epileptic patients.

Allopregnanolone (CAS Reg. No. 516-54-1, 3α,5α-tetrahydroprogesterone) is an endogenous progesterone derivative with anti-convulsant activity.

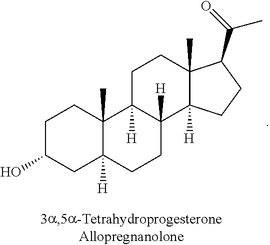

Allopregnanolone has a relatively short half-life, 45 minutes in human plasma. In addition to its efficacy in treating seizures, allopregnanolone is being evaluated for use in treating neurodegenerative diseases including Alzheimer's disease, Parkinson's disease, Huntington's disease, and amyotrophic lateral sclerosis and for treating lysosomal storage disorders characterized by abnormalities in cholesterol synthesis, such as Niemann Pick A, B, and C, Gaucher disease, and Tay Sachs disease. (See U.S. Pat. No. 8,604,011, which is hereby incorporated by reference for its teachings regarding the use of allopregnanolone for treating neurological disorders.)

Alphaxalone, also known as alfaxalone, (CAS Reg. No. 23930-19-0, 3α-hydroxy-5α-pregnan-11,20-dione) is a neurosteroid with an anesthetic activity. It is used as a general anaesthetic in veterinary practice. Anaesthetics are frequently administered in combination with anti-convulsants for the treatment of refractory seizures. An injectable nanoparticle neurosteroid dosage form containing alphaxalone alone or in combination with either ganaxolone or allopregnanolone is within the scope of this disclosure.

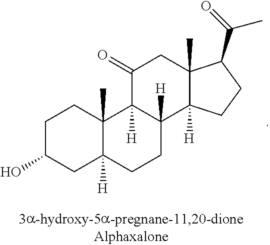

Alphadolone, also known as alfadolone, (CAS Reg. No. 14107-37-0, 3α,21-dihydroxy-5α-pregnan-11,20-dione) is a neurosteroid with anaesthetic properties. Its salt, alfadolone acetate is used as a veterinary anaesthetic in combination with alphaxalone.

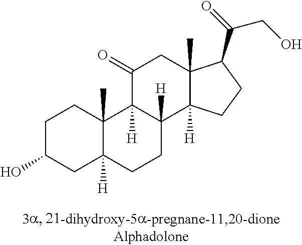

Additional neurosteroids that may be used in the injectable nanoparticle neurosteroid formulation of this disclosure include formulations include hydroxydione (CAS Reg. No. 303-01-5, (5β)-21-hydroxypregnane-3,20-dione), minaxolone (CAS Reg. No. 62571-87-3, 2β,3α,5α,11α)-11-(dimethylamino)-2-ethoxy-3-hydroxypregnan-20-one), pregnanolone (CAS Reg. No. 128-20-1, (3α,5β)-d-hydroxypreganan-20-one), renanolone (CAS Reg. No. 565-99-1, 3α-hydroxy-5β-pregnan-11,20-dione), isopregnanolone (CAS Reg. No. 516-55-2, 3β-Hydoxy-5α-pregnan-20-one) or tetrahydrocorticosterone (CAS Reg. No. 68-42-8, 3α,5α-pregnan-20-dione).

In certain embodiments the neurosteroid is a compound of Formula I, as shown in the SUMMARY section, or a pharmaceutically acceptable salt of such a compound. In certain embodiments the neurosteroid is ganaxolone. In other embodiments the neurosteroid is allopregnanolone, alphaxalone, minaxolone, tetrahydrodeoxycorticosterone, etiocholanone, dehydroepiandrosterone, or pregnanolone, or a pharmaceutically acceptable salt of any of the foregoing.

In certain embodiments the neurosteroid is allopregnanolone, ganaxolone, alphaxalone, alphadolone, hydroxydione, minaxolone, pregnanolone, acebrochol, isopregnanolone, or tetrahydrocorticosterone.

The disclosure includes compounds of Formula I as disclosed in the SUMMARY section in which the neurosteroid is compound of Formula I, where any of the following conditions for the variables (e.g. R¹-R¹¹) are met. All definitions of the variables used in this disclosure can be combined so long as a stable compound of Formula I results.

R¹ is methyl, —CH₂Br, or —CH₂OH.

R¹ is a group of the formula

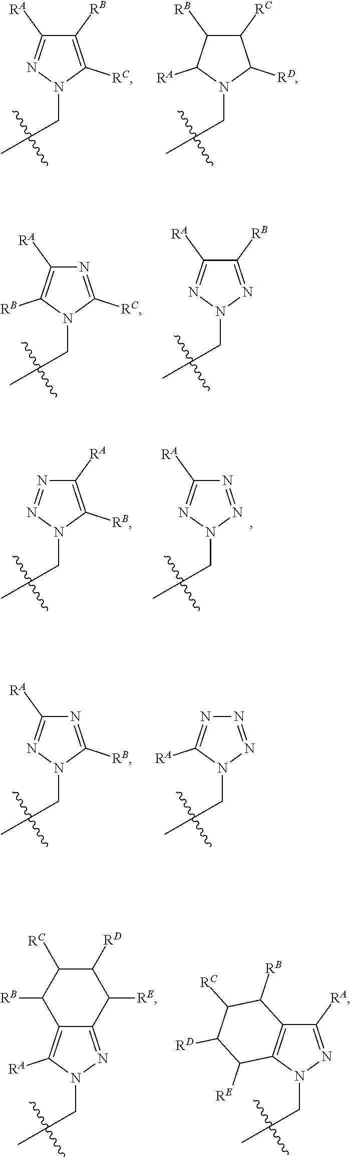

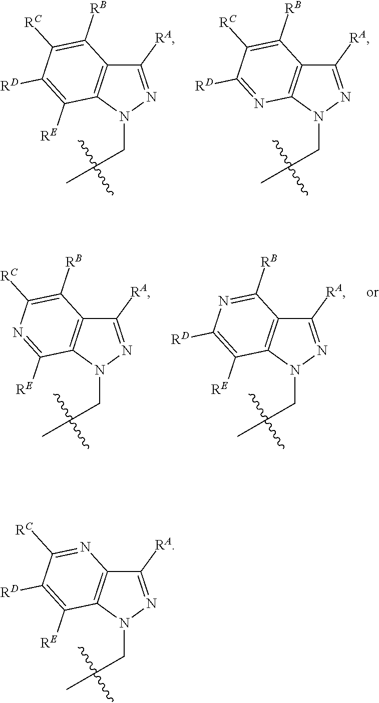

each instance of R^{A}, R^{B}, R^{C}, R^{D}, and R^{E} is, independently, hydrogen, halogen, —NO₂, —CN, —OR^{GA}, —N(R^{GA})₂, —C(═O)R^{GA}, —C(═O)OR^{GA}, —OC(═O)R^{GA}, —OC(═O)OR^{GA}, —C(═O)N(R^{GA})₂, —N(R^{GA})C(═O)R^{GA}, —OC(═O)N(R^{GA})₂, —N(R^{GA})C(═O)OR^{GA}, —N(R^{GA})C(═O)N(R^{GA})₂, —SR^{GA}, —S(O)R^{GA}, e.g., —S(═O)R^{GA}, —S(═O)₂R^{GA}, —S(═O)₂OR^{GA}, —OS(═O)₂R^{GA}, —S(═O)₂N(R^{GA})₂, —N(R^{GA})S(═O)₂R^{GA}, optionally substituted alkyl, optionally substituted C₃-C₆ carbocylyl, or optionally substituted 3- to 6-membered heterocylyl; and where instance of R^{GA} is independently hydrogen, optionally substituted alkyl, optionally substituted C₃-C₆ carbocylyl, optionally substituted 3- to 6-membered heterocylyl, optionally substituted aryl, optionally substituted heteroaryl, an oxygen protecting group when attached to oxygen, nitrogen protecting group when attached to nitrogen, or two R^{GA} groups are taken with the intervening atoms to form a substituted or unsubstituted heterocylyl or heteroaryl ring.

In certain embodiments R^{A}, R^{B}, R^{C}, R^{D} and R^{E} are independently chosen from hydrogen, halogen, cyano, methyl, methyoxy, ethyl, ethoxy, C₁-C₂haloalkyl, and C₁-C₂haloalkoxy.

In certain embodiments, R¹ is a group of the formula

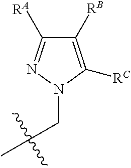

and R^{A}, R^{B}, and R^{C}, are all hydrogen or R^{A} and R^{C} are hydrogen and R^{B} is cyano.

The disclosure pertains to compounds and salts of Formula I having any of the above R¹ values where R² is methyl, R³ is hydrogen, R⁴ and R^{4a} are both hydrogen or are taken together to form an oxo group; R⁶ is hydrogen, R⁷ is hydrogen, R⁸ is hydrogen or methyl, R⁹ is hydroxyl, or R⁸ and R⁹ are taken together to form an oxo group, and R¹⁰ and R^{10A} are both hydrogen.

In certain embodiments [symbol] is also a single bond.

In certain embodiments a Formula I is represented by one of the following substructures:

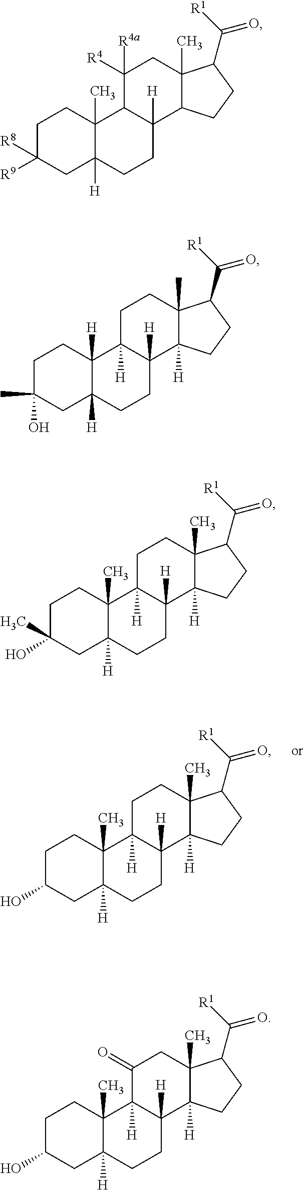

### Methods of Administration

The disclosure provides a method of administering a ganaxolone formulation to elicit EEG burst suppression/EEG suppression. Methods of administration include intravenous, intramuscular, oral, and subcutaneous administration. For example, burst suppression may be effected by continuous intravenous administration of an injectable neurosteroid formulation comprising neurosteroid nanoparticles having a D50 of less than 2000 nm and a polymeric surface stabilizer. Clinical studies in healthy volunteers demonstrated that a continuous IV infusion of a ganaxolone formulation reduces bispectral index (BIS), a measure of anesthesia that represents a slowing of EEG. BIS is not equivalent to burst suppression, but is somewhat indicative of the lowest dose at which burst suppression may be induced. Doses of 4 mg/hr have been found to produce BIS changes.

Methods of administration also include repeated intravenous administration.

Other embodiments include intravenously, intramuscularly, orally or subcutaneously administering sequential bolus doses of a neurosteroid formulation comprising 0.1 mg to 100 mg, 0.1 mg to 50 mg neurosteroid per 1 kg of patient body weight, or 0.5 mg of neurosteroid per 1 kg of body weight with an interval of less than 30 minutes between two consecutive doses. Many neurosteroids, including ganaxolone and allopregnanolone, exhibit poor oral availability and achieving effective brain levels has proved challenging. It has been discovered that injectable neurosteroid formulations comprising nanoparticles having a D50 of less than 2 m (2000 nm) can provide surprisingly high brain levels of neurosteroid. The nanoparticles comprise the neurosteroid and a polymeric surface stabilizer such as hydroxylethyl starch, dextran, or povidone, and optionally an ionic or nonionic surfactant as an additional surface stabilizer. The ability of these injectable nanoparticle neurosteroid formulations to provide such high levels of neurosteroid makes them particularly well suited for inducing burst suppression and effecting anesthesia in patients. In an embodiment, the intravenous administration may be an injection, which may be given at intervals of less than 30 minutes between two consecutive injections. The neurosteroid formulation may be injected every 25 minutes, every 20 minutes, every 15 minutes, every 10 minutes, every 5 minutes, every 4, minutes, every 3 minutes, every 2 minutes, and every 1 minute, and can be followed by a continuous infusion to maintain the EEG state. For example, the neurosteroid formulation may be injected every 3 minutes. Administration may also be by continuous IV infusion at dose infusion rates of 0.1 mg to 3 mg per kilogram of body weight per minute.

The administration of the neurosteroid formulation as described in the preceding paragraph may elicit burst suppression/EEG suppression patterns, which may be observed by conducting electrophysiology experiments. Modern scientific experiments strongly support a hypothesis that anesthetics bring about oscillations that modulate or disrupt the oscillations normally produced by the brain (Purdon, P. L., Sampson, A., Pavone, K. J., Brown, E. N., Anesthesiology, 2015 October; 123 (4): 937-960). These spectral changes can be readily seen in the electroencephalogram. Administration of the anesthetics can induce various behavioral and neurophysiological states characterized by different electroencephalogram waveforms. These depth-of-anesthesia states may include the awake state, paradoxical excitation, a sedative state, slow and alpha oscillation anesthetic state, slow oscillation anesthetic state, burst suppression, and isoelectric state. Among them, burst suppression represents a state of unconsciousness and profound brain inactivation, which is characterized by periods of electrical activity alternating with periods of isoelectricity (electrical silence). In the spectrogram, burst suppression can be seen as vertical lines separated by periods of brain inactivity. Upon administration of many anesthetics, burst suppression is attained through the intermediate states. However, it was shown that upon administration of propofol as an induction bolus, patients can enter burst suppression directly from the awake state.

Burst suppression can be brought about by hypothermia for surgeries requiring total circulatory arrest. Alternatively, it can be induced by administering anesthetics in the intensive care unit for cerebral protection to treat intracranial hypertension or to treat status epilepticus. The anesthetic administration is also known as a medically-induced coma. When a patient is in burst suppression, an increase in the anesthetic dose results in the increase of the length of the suppression periods between the bursts. Brain suppression has also been observed in other conditions accompanied by profound brain inactivation, such as coma, or in individuals with compromised brain development. This data suggest that different mechanisms may be responsible for attaining this state.

Burst suppression may be quantified by using a burst suppression ratio or a burst suppression probability. The burst suppression ratio is a number between 0 and 1, which measures the fraction of time in a given time interval that the electroencephalogram is suppressed. The burst suppression probability is an instantaneous probability of the brain to be in a state of suppression, which can be computed using state-space methods, and which can be used to track burst suppression in real time as well as to implement control systems for a medical coma. Both burst suppression ratio and burst suppression probability have been used to assess the depth of anesthesia.

The characteristic pattern of burst suppression is a consequence of extracellular calcium depletion, which is restored by the action of neurons. In the spectrogram, bursts are alternated by suppression periods which are caused by depletion of extracellular cortical calcium ions to the levels that inhibit synaptic transmission. During suppression, neurons restore the calcium ion concentrations to normal levels. As the dose of anesthetic is increased, the brain becomes more inactive, and burst periods become shorter while suppression periods become longer. Further increase of the anesthetic dose in a patient normally leads to the isoelectric state.

Because of its characteristics, the burst suppression pattern can be used to evaluate a level of a coma in a patient. The pattern can also be used to assess the ability of anesthetic arousal agents to induce emergence of a patient from a coma.

In an embodiment, the EEG signal may be measured for 10 hours, 9 hours, 8 hours, 7 hours, 6 hours, 5 hours, 4 hours, 3 hours, 2 hours, or 1 hour. For example, the EEG signal may be measured continuously for 6 hours. In an embodiment, burst suppression may be first detected after the second administration, third administration, fourth administration, fifth administration, sixth administration, seventh administration, eighth administration, ninth administration, and tenth administration of neurosteroid. Thus, to elicit the burst suppression effect, at least two, at least three, at least four, at least five, at least six, at least seven, at least eight, or at least nine neurosteroid bolus doses may be administered. For example, no more than ten neurosteroid doses may be administered.

In an embodiment, a cumulative bolus dose of neurosteroid is at least 0.1 mg/kg, 0.5 mg/kg, 1 mg/kg, 3 mg/kg, 6 mg/kg, 9 mg/kg, 12 mg/kg, 15 mg/kg, 18 mg/kg, 21 mg/kg, 24 mg/kg, 27 mg/kg, 30 mg/kg, 35 mg/kg, 40 mg/kg, 45 mg/kg, or 50 mg/kg. For example, in some embodiments the cumulative bolus dose of neurosteroid is at least 30 mg/kg. The disclosure includes embodiments in which administration of the neurosteroid, does not produce full anesthesia is the patient. The disclosure also includes embodiments in which burst suppression is achieved and full anesthesia is effected in the patient.

A concentration of neurosteroid in the formulation may be 0.1 mg/mL to 15 mg/mL, for example, 1 mg/mL to 10 mg/mL.

In another embodiment, the neurosteroid formulation may be an injectable neurosteroid formulation comprising nanoparticles having a D₅₀ of less than 2000 nm, the nanoparticles comprising:

a) neurosteroid; and

b) at least one polymeric surface stabilizer.

The injectable neurosteroid nanoparticle formulations may contain neurosteroid at a concentration of 0.25 mg/mL, 0.5 mg/mL, 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 10 mg/mL, 11 mg/mL, 12 mg/mL, 13 mg/mL, or 15 mg/mL, 20 mg/mL, 25 mg/mL, 30 mg/mL, 35 mg/mL, 40 mg/mL, 45 mg/mL, or 50 mg/mL. All ranges including any two of the foregoing concentrations of neurosteroid as endpoints are also included in the disclosure. For example, the disclosure includes neurosteroid nanoparticle formulations containing from 0.5 mg/mL to 15 mg/mL, 1.0 mg/mL to 10 mg/mL, 2.0 mg/mL to 8.0 mg/mL, or 4.0 mg/mL to 8.0 mg/mL neurosteroid.

The nanoparticles may include neurosteroid and a surface stabilizer, such as either hydroxyethyl starch, povidone, or dextran, wherein a weight to weight ratio of neurosteroid to surface stabilizer is 10:1 to 0.2:1, or 5:1 to 0.2:1, or 4:1 to 1:1, or 3.5:1 to 3:1, or 3.3:1. The disclosure includes embodiments in which the injectable neurosteroid nanoparticle formulation additionally comprises a buffer. In certain embodiments, the buffer is a phosphate buffer. In certain embodiments, the buffer is a phosphate buffered saline.

In certain embodiments, the surface stabilizer may be a blood replacer, such as a blood volume expander. In other embodiments, the surface stabilizer is either hydroxyethyl starch, dextran, or povidone. Hydroxyethyl starch is used as a blood volume expander in patients suffering from severe blood loss. Grades of hydroxyethyl starch suitable for use in the neurosteroid nanoparticles include 130/0.4 (CAS Reg. No. 9005-27-0). In certain embodiments, the surface stabilizer is dextran. Dextran is a single chain branched glucan having chains of varying lengths. Like hydroxyethyl starch, dextran is also used as a blood volume expander. Dextrans are classified according to MW. Dextrans having molecular weights from 40 kD to 75 kD have been used as blood volume expanders. Suitable dextrans for intravenous use include Dextran 40, Dextran 60, Dextran 70, and Dextran 75. In certain embodiments, the surface stabilizer is a dextran having a molecular weight from 40 kD to 75 kD. In certain embodiments, the surface stabilizer is Dextran 70. Povidone, also known as polyvinylpyrrolidone, is another approved plasma expander. Povidone includes PLASDONE Povidone from Ashland.

Other excipients useful as surface stabilizers for the injectable neurosteroid nanoparticle formulation include human serum albumin, hydrolyzed gelatin, polyoxyethylene castor oil, and polyoxyethylene hydrogenated castor oil. The injectable neurosteroid nanoparticle injectable formulation includes a surfactant.

Surfactants include compounds such as lecithin (phosphatides), sorbitan trioleate and other sorbitan esters, polyoxyethylene sorbitan fatty acid esters (e.g., the commercially available TWEENS such as polyoxyethylene sorbitan monolaurate (TWEEN 20) and polyoxyethylene sorbitan monooleate (TWEEN 80) (ICI Speciality Chemicals)); poloxamers (e.g., poloxamer 188 PLURONIC F68 and poloxamer 338 (PLURONIC F108), which are block copolymers of ethylene oxide and propylene oxide), lecithin, sodium cholesterol sulfate or other cholesterol salts, and bile salts, such as sodium deoxycholate. Additional bile salts that may be used as surfactants include sodium cholate, sodium glycholate, salts of deoxycholic acid, salts of glycholic acid, salts of chenodeoxycholic acid, and salts of lithocholic acid.

The disclosure includes neurosteroid nanoparticles having a volume weighted median diameter (D₅₀) of from 50 nm to 2000 nm, 50 nm to 500 nm, 10 nm to 350 nm, or having a D₅₀ of from 50 nm to 300 nm, or having a D₅₀ of from 100 nm to 250 nm, or having a D₅₀ of 150 nm to 220 nm, or having a D₅₀ of less than 2000 nm, less than 500 nm, of less than 350 nm, less than 300 nm, less than 250 nm, or less than 200 nm.

In one aspect the neurosteroid nanoparticles have at least one of the following properties: (a) greater than 90% of neurosteroid by weight is in the form of submicron particle having an effective size of 50 nm to 300 nm; (b) at least 20% of neurosteroid by weight is in the form of an amorphous powder; (c) at least 50% of neurosteroid by weight is in the form of a crystalline powder of a single polymorph; (d) at least 50% of neurosteroid is in the form of a semi-crystalline powder; (e) neurosteroid is in the form of particles wherein at least 50%, or at least 60%, or at least 70%, or at least 80%, or at least 90% of the particles by weight have an effective size less than 300 nm; (f) neurosteroid is in the form of particles wherein at least 50% of the particles by weight have an effective size of less than 250 nm; (g) neurosteroid is in the form of particles having a D₅₀ of 50 nm to 200 nm, wherein the particle size distribution is described by a three-slice model in which a certain percentage has an effective particle size by weight between 10 nm and 100 nm, a certain percentage has an effective particle size by weight between 100 nm and 200 nm, and a certain percentage has an effective particle size by weight above 200 nm, and further wherein the three-slice model is identified as x %/y %/z %, respectively (e.g., 4030%/30%/30%); (p) neurosteroid has a three-slice distribution selected from the group 40%/30%/30%, 50%/30%/20%, 60%/30%/i 0%, 40%/40% a/20%, 50%/40%/10%, 70%/20%/10%, 50%/45%/5%, 70%/25%/5%, 60%/35%/5%, 80%/15%/5%, 70%/30%/0%, 60%/40%/0%, 90%/10%/0%, and 100%/0%/0%; (h) neurosteroid is in the form of particles, wherein standard deviation of the particle size distribution divided by the volume-weighted mean diameter is less than 30%, less than 25%, less than 20%, less than 15%, or less than 10%. In alternative embodiments, neurosteroid in the composition has at least two of the aforementioned properties; at least three of the aforementioned properties; at least four of the aforementioned properties; or at least five of the aforementioned properties.

The neurosteroid nanoparticles may be prepared by grinding. Grinding can take place in any suitable grinding mill. Suitable mills include an air jet mill, a roller mill, a ball mill, an attritor mill, a vibratory mill, a planetary mill, a sand mill and a bead mill, A high energy media mill is preferred when small particles are desired. The mill can contain a rotating shaft.

The preferred proportions of the grinding media, neurosteroid, the optional liquid dispersion medium, and dispersing, wetting or other particle stabilizing agents present in the grinding vessel can vary within wide limits and depends, for example, the size and density of the grinding media, the type of mill selected, the time of milling, etc. The process can be carried out in a continuous, batch or semi-batch mode. In high energy media mills, it can be desirable to fill 80-95% of the volume of the grinding chamber with grinding media. On the other hand, in roller mills, it frequently is desirable to leave the grinding vessel up to half filled with air, the remaining volume comprising the grinding media and the liquid dispersion media, if present. This permits a cascading effect within the vessel on the rollers which permits efficient grinding. However, when foaming is a problem during wet grinding, the vessel can be completely filled with the liquid dispersion medium or an anti-foaming agent may be added to the liquid dispersion.

The attrition time can vary widely and depends primarily upon the drug, mechanical means and residence conditions selected, the initial and desired final particle size and so forth.

After attrition is completed, the grinding media is separated from the milled neurosteroid particulate product (in either a dry or liquid dispersion form) using conventional separation techniques, such as by filtration, sieving through a mesh screen, and the like.

In one aspect, the grinding media comprises beads having a size ranging from 0.05-4 mm, preferably 0.1-0.4 mm. For example, high energy milling of neurosteroid with yttrium stabilized zirconium oxide may produce 0.4 mm beads for a milling residence time of 25 minutes to 1.5 hours in recirculation mode at 2500 revolutions per minute (RPM). In another example, high energy milling may involve neurosteroid with 0.1 mm zirconium oxide balls for a milling residence time of 2 hours in batch mode. Additionally, the milling temperature should not exceed 50° C. as the viscosity of the suspension may change dramatically. The milling concentration is from 1% to 40% neurosteroid by weight. In an embodiment, the concentration is 25% neurosteroid by weight. In one embodiment, the milling media contains at least one agent to adjust viscosity so that the desired particles are suspended evenly, and a wetting and/or dispersing agent to coat the initial neurosteroid suspension so a uniform feed rate may be applied in continuous milling mode. In another embodiment, batch milling mode is utilized with a milling media containing at least one agent to adjust viscosity and/or provide a wetting effect so that neurosteroid is well dispersed amongst the grinding media.

The injectable neurosteroid nanoparticle formulations may also include an acid or base buffer to adjust pH to desired levels. In some embodiments, the desired pH is 2.5-11.0, 3.5-9.0, or 5.0-8.0, or 6.0-8.0, or 7.0-7.6, or 7.4. Examples of acid buffers useful in the injectable neurosteroid nanoparticle formulation include oxalic acid, maleic acid, fumaric acid, lactic acid, malic acid, tartaric acid, citric acid, benzoic acid, acetic acid, methanesulfonic acid, histidine, succinic acid, toluene sulfonic acid, benzene sulfonic acid, ethane sulfonic acid and the like. Acid salts of the above acids may be employed as well. Examples of base buffers useful in the formulation include carbonic acid and bicarbonate systems such as sodium carbonate and sodium bicarbonate, and phosphate buffer systems, such as sodium monohydrogen phosphate and sodium dihydrogen phosphate. The concentration of each component of a phosphate buffer system will be from 10 mM to 200 mM, or from 20 mM to 150 mM, or from 50 mM to 100 mM.

The disclosure includes embodiments in which the pH of the neurosteroid nanoparticle formulation is about 7.4.

The formulation may contain electrolytes, such as sodium or potassium. The disclosure includes embodiments in which the formulation is from 0.5% to 1.5% sodium chloride (saline).

The formulation may contain tonicity adjusting agents so that it is isotonic with human plasma. Examples of tonicity adjusting agents useful in the formulation include, but are not limited to, dextrose, mannitol, sodium chloride, or glycerin. In certain embodiments, the tonicity agent is 0.9% sodium chloride.

The injectable neurosteroid nanoparticle formulations may contain any pharmaceutically acceptable excipient compatible with neurosteroid and capable of providing the desired pharmacological release profile for the dosage form. Excipients include, for example, suspending agents, surfactants, solubilizers, stabilizers, lubricants, wetting agents, anti-foaming agent, diluents, and the like. Pharmaceutically acceptable excipients may comprise, but are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

Suitable antifoaming agents include dimethicone, myristic acid, palmitic acid, and simethicone.

The injectable neurosteroid nanoparticle formulation may also contain a non-aqueous diluent such as ethanol, one or more polyol (e.g., glycerol, propylene glycol), an oil carrier, or any combination of the foregoing.

The injectable neurosteroid nanoparticle formulation may additionally comprise a preservative. The preservative may be used to inhibit bacterial growth or prevent deterioration of the active agent. Preservatives suitable for parenteral formulations include ascorbic acid, acetylcysteine, benzalkonium chloride, benzethonium chloride, benzoic acid, benzyl alcohol, chlorbutanol, chlorhexidene, m-cresol, 2-ethoxyethanol, human serum albumin, monothioglycerol, parabens (methyl, ethyl, propyl, butyl, and combinations), phenol, phenylmercurate salts (acetate, borate nitrate), sorbic acid, sulfurous acid salts (bisulfite and metabisulfite), and thimerosal. In certain embodiments the preservative is an antioxidant such ascorbic acid, glutathione, or an amino acid. Amino acids useful as antioxidants include methionine, cysteine, and L-arginine.

In an embodiment, the neurosteroid nanoparticles may have a D₅₀ of less than 500 nm.

The disclosure also provides a method for detecting efficacious dose-levels of neurosteroid for treatment of an epileptic condition or for effecting anesthesia. After administration of an efficacious dose, electroencephalographic burst suppression in the electroencephalography pattern is detected. The efficacious dose is subsequently maintained in the patient to remedy the epileptic condition or maintain anesthesia.

In an embodiment the neurosteroid is administered intravenously as a continuous infusion to effect burst suppression or determine the neurosteroid dose needed to effect burst suppression. According to the method the neuorosteroid can be administered at a rate of 1 mg/hr to 1000 mg/hr, 1 mg/hr to 500 mg/hr, 5 mg/hr to 300 mg/hr, 10 mg/hr to 200 mg/hr, or 20 mg/hr to 150 mg/hr, 5 mg/hr to 150 mg/hr, 5 mg/hr to 100 mg/hr, at least 5 mg/hr, at least 10 mg/hr, at least 20 mg/hr, or at least 50 mg/hr. The neurosteroid can also be administered at a rate of 0.01 mg/kg/hr to 15 mg/kg/hr, 0.5 mg/kg/hr to 10 mg/kg/hr, 0.05 to 5 mg/kg/hr, 0.1 mg/kg/hr to 3.5 mg/kg/hr, or 0.2 mg/kg/hr to 2.5 mg/kg/hr, or at least 0.5 mg/kg/hr, at least 1 mg/kg/hr, at least 2 mg/kg/hr, or at least 5 mg/kg/hr.

Neurosteroid can be administered until target plasma levels are achieved. In certain embodiment neurosteroid needed to obtain a neurosteroid concentration of at least 10 ng/ml, at least 20 ng/ml, at least 50 ng/ml, at least 100 ng/ml, at least 200 ng/ml, or at least 500 ng/ml is administered. In certain embodiments neurosteroid needed to achieve a neurosteroid plasma level of 10 ng/ml to 5000 ng/ml, 10 ng/ml to 1000 ng/ml, 10 ng/ml to 500 ng/ml or 10 ng/ml to 300 ng/ml is administered.

In an embodiment bolus doses of neurosteroid doses are used to effect burst suppression or determine the dose necessary to produce burst suppression. According to the method, sequential bolus doses of a formulation comprising 0.5 mg of neurosteroid per 1 kg of body weight with an interval of less than 30 minutes between two consecutive doses are first administered, intravenously, intramuscularly, or subcutaneously to a patient, to treat an epileptic condition. To effect anesthesia the efficacious dose is higher for example 0.1 mg to 50 mg/kg, or 0.5 mg/kg, or 1 mg/kg, or 5 mg/kg, 10 mg/kg, 15 mg/kg, 20 mg/kg, 30 mg/kg, 40 mg/kg, or 50 mg/kg. An electroencephalography pattern in a brain of the patient is then continuously measured.

To elicit the burst suppression effect, at least two, at least three, at least four, at least five, at least six, at least seven, at least eight, or at least nine neurosteroid bolus doses may be administered. For example, no more than ten neurosteroid doses may be administered.

In an embodiment, a cumulative bolus dose of neurosteroid may not exceed 9 mg/kg, 12 mg/kg, 15 mg/kg, 18 mg/kg, 21 mg/kg, 24 mg/kg, 27 mg/kg 30 mg/kg, 40 mg/kg, or 50 mg/kg. For example, the cumulative bolus dose of neurosteroid in certain embodiments, to effect anesthesia a cumulative dose of at least 30 mg/kg.

The neurosteroid formulation may be administered every one minute, every two minutes, every three minutes, every four minutes, every five minutes, every ten minutes, every fifteen minutes, every twenty minutes, or every twenty five minutes. For example, the neurosteroid formulation may be administered every three minutes. The cumulative administration of the neurosteroid formulation produces full anesthesia in a patient.

The present inventive concept is further illustrated by the following examples. These examples are illustrative only and do not limit the scope of the present inventive concept.

### EXAMPLES

### Example 1. Preparation of Injectable Ganaxolone Captisol Formulation

The following formulation is used as a positive control. To prepare injectable solutions, excess ganaxolone is added to an aqueous 400 mg/mL CAPTISOL solution. The solution is shaken at least overnight and filtered through a 0.45 micron filter. Ganaxolone concentration of the filtered solution is determined by HPLC. The ganaxolone/CAPTISOL solution (7.68 mg/mL ganaxalone in 400 mg/mL aqueous CAPTISOL) is diluted in saline to obtain 3.84 mg/mL, 0.77 mg/mL and 0.39 mg/mL ganaxolone solutions in 0.9% saline. All solutions were clear and free from any visible precipitation. The ganaxolone solutions remained free of any visible precipitation after freezing and thawing.

### Example 2. Injectable Ganaxolone-Captisol Solution (5 mg/ml)

This formulation is also used a positive control. Ganaxolone (0.50 g) was mixed manually using a spatula with a small amount (approximately 20 mL) of 30% w/v CAPTISOL solution in sterile water for injection to form a uniform paste. Additional amounts (approximately 40 mL) of 30% w/v CAPTISOL solution was then added to obtain a slurry. The suspension was stirred using a magnetic stir bar for 20 min. It was sonicated using a probe sonicator for 2 hours (h). While sonicating, an additional 30% w/v CAPTISOL solution was added until total amount of the CAPTISOL solution reached 99.58 mL. The stirred formulation was then heated at 68.5° C. for 2.5 hours to obtain a solution. Heat was removed and the solution was stirred at room temperature for approximately 2 h. Volume lost due to evaporation was replenished with water. The clear solution was sterile filtered through a 0.2 μm Nylon membrane filter.

### Example 3. Preparation of Ganaxolone Nanosuspension (10% wt) Via Wet Bead Milling

An aqueous slurry (250 g) containing ganaxolone (25 g), hydroxyethyl starch (7.5 g), sodium deoxycholate (0.5 g) and 30% simethicone (1 drop) was milled using a Netzsch Mill (Minicer) with 0.3 mm YTZ beads (Yttrium stabilized grinding media, Tosoh Corporation, Japan, ZrO₂+HfO₂ (95 wt % (weight %)), Y₂O₂ (5 wt %)). Two additional portions of solid sodium deoxycholate (0.5 g each) were added at 100 and 130 min after milling had started. The particle size of the milled slurry was measured using a Horiba LA-910 laser diffraction particle size analyzer. After 170 minutes of milling, D50 was 192 nm (188 nm after 1 min sonication). At this point, milling was stopped and the milled slurry was kept at room temperature overnight. The next morning, milling was resumed until the total milling time had reached 320 minutes, at which point D50 was 167 nm (169 nm after 1 min sonication). The D50 particle size was measured on a Horiba 910 Laser Light Scattering instrument.

### Example 4. Ganaxolone Nanosuspension Containing Poloxamer 188

A KDL Bachofen Mill was configured with the batch chamber attachment (approx. 350 ml) and the 96 mm polyurethane rotor attached to the shaft. Next, 265 ml of 0.3 mm ytria-zirconia beads were added dry to the chamber, followed by 176.7 g of the ganaxolone (GNX) slurry. Slowly, over 15 minutes, the ganaxolone slurry was added to the stabilizer solution containing Pluronic F-68 (Poloxamer 188) with sustained stirring. The mixture was stirred slowly overnight. The slurry was milled at Speed 1 (1500 rpm) with intermittent measurement of particle size. After 90 min, the D50 particle size was determined to be 378 nm. The D50 measurement was measured on a Horiba 910 Laser Light Scattering instrument.

Stabilizer solution

| | | |
|---|---|---|
| Pluronic F-68 | 27.0 | g |
| Sodium deoxycholate | 2.7 | g |
| Simethicone emulsion 30% | 0.2 | g |
| Water (DI) | to 200 | g |

Ganaxolone Slurry

| | | |
|---|---|---|
| Ganaxolone | 50 | g |
| Stabilizer solution | 150 | g |

Final Milling Composition (wt %)

| | |
|---|---|
| Ganaxolone | 25% |
| Pluronic F-68 | 10% |
| Deoxycholate | 1% |

### Example 5. Ganaxolone Nanosuspension Containing 12.5% Poloxamer 188 and Dextran

The KDL Bachofen mill was configured with the batch chamber attachment (approx. 350 ml) and the 96 mm polyurethane rotor attached to the shaft. Next, 300 ml of 0.1 mm yttria-zirconia beads were added dry to the chamber, followed by 176.5 gm of the Ganaxolone (GNX) milling suspension. The ganaxolone milling suspension was prepared by combining the dextran, Pluronic F-68, sodium deoxycholate, and simethicone emulsion ingredients with stirring, and then adding the ganaxolone last with stirring. The suspension stirred for 1.5 hr. The suspension (176.5 gm was added to the batch chamber and the mill started at Speed setting 1. The slurry was milled for 60 minutes and the D50 particle size was measured after 20, 40, 50, and 60 minutes of milling.

Ganaxolone Milling Suspension

| | | |
|---|---|---|
| Dextran (40K mol. wt.) | 10.0 | g |
| Pluronic F-68 | 25.0 | g |
| Sodium deoxycholate | 0.5 | g |
| Simethicone emulsion 30% | 0.2 | g |
| Ganaxolone | 20.0 | g |
| Water (DI) | to 200 | g |

Final Milling Composition (wt %)

| | |
|---|---|
| Ganaxolone | 20% |
| Dextran | 5% |
| Pluronic F-68 | 25% |
| Sodium Deoxycholate | 0.25% |

### Example 6. Additional Injectable Nanoparticle Formulations

TABLE 2 shows the compositions of formulation I-VI which are suitable for use in the burst suppression methods of this disclosure's experiments. The polymers used in formulation I-V are I, Plasdone C17; II, hydroxyethyl starch 130/0.4; III, Dextran 70, IV, Plasdone C12, V, hydroxyethyl starch 130/0.4. The API is ganaxolone for all formulations.

TABLE 2
Composition of formulations I-V

| | Formulation | | | | |
|---|---|---|---|---|---|
| | I | II | III | IV | V |
| Ganaxolone | 5.43% | 5.42% | 5.57% | 5.50% | 5.50% |
| Polymer | 5.43% | 5.42% | 5.57% | 5.50% | 11.00% |
| Na Deoxycholate | 0.65% | 0.65% | 0.67% | 0.66% | 0.66% |
| Simethicone 30% emulsion | 0.03% | 0.03% | 0.03% | 0.03% | 0.03% |
| Deionized water | 88.46% | 88.46% | 88.16% | 88.31% | 82.81% |
| Total | 100.00% | 100.00% | 100.00% | 100.00% | 100.00% |

### Example 7. Burst Suppression Studies

### Animals

Male Sprague-Dawley rats were used for the studies. Rats were surgically implanted with EEG electrodes and jugular vein catheters. The mean weight at the time of recording was 321±3 g (269-351 g).

EEG electrodes were surgically implanted in anesthetized rats. Animals were also treated with an anti-inflammatory analgesic (RIMADYL, (carprofen)) prior to surgery and a subcutaneous local anesthetic. Stainless steel screw electrodes chronically were implanted in the skull (0-80×¼″, Plastics-One, Roanoke, Va.) such that the ends of the screws were flush with the inner skull surface. One electrode was located 3.0 mm anterior to bregma and 2 mm to the left of midline, and the second 4.0 mm posterior to bregma and 2.5 mm to the right of midline. The skull surface around the electrodes was sealed with super glue and dental acrylic, and the EEG electrode lead wires were inserted into a plastic pedestal mounted on the skull using dental acrylic. Wound edges were treated with triple antibiotic cream (bacitracin zinc, neomycin sulfate, polymyxin B sulfate; Walgreens). Following surgery, the animals were administered antibiotic (ampicillin, 50 mg/kg IP at 0.4 mL/kg) and also received a chewable dose (˜10 mg) of Rimadyl. The animals were allowed to recover from EEG surgery for 1 week.

Rats were implanted with jugular-vein catheters (JVC) one week following EEG surgery. Rats were administered an anti-inflammatory/analgesic and then anesthetized and placed in a supine position. An incision was made in the skin on the right ventrolateral aspect of the neck to expose the external jugular vein which was then dissected free of surrounding fascia. A ligature was tied around the vein anteriorly to occlude blood flow returning to the heart. A second ligature was loosely tied around the vein posteriorly to create tension on the vessel during venotomy and catheter insertion. After incising the vein, a PE catheter (3 Fr) was inserted into the vein and advanced approximately 30 mm towards the heart, positioning the tip at the junction of the precava and right atrium. After confirming patency of the catheter by blood withdrawal, the posterior ligature was tied off, and the catheter flushed with a “heparin-lock” solution (5 units heparin/ml saline) and plugged with a sterilized stainless steel pin. The catheter was then exteriorized by tunneling through the subcutaneous tissue to exit posterior to the head between the shoulder blades. Lastly, after suturing the catheter to the skin, all wounds were closed using sutures or wound clips. Immediately following surgery, the animals were awakened to assess catheter patency. Animals also received a chewable dose (˜10 mg) of Rimadyl following surgery to minimize pain and inflammation.

Animals were allowed a 1-week recovery time following JVC surgery. Jugular vein catheters were flushed daily with a 0.1 mL of heparin solution (5 units/mL) to maintain patency. On the day of surgery, the flushing solution contained ampicillin sulbactam (50 mg/kg). If any catheters became difficult to flush, the animal number and date was recorded so that the animal could be excluded or assigned to a vehicle group if possible. Anesthesia is measured by tail pinch—scored 0 to 3, with 3 representing anesthesia. EEG Recording Procedure

For EEG recording, each rat was dosed with LiCi and placed into a recording container (30×30×30 cm with a wire-mesh grid top) the evening prior to recording. Animals were not fasted, and had ad libitum access to food and water prior to scopolamine administration, at which time food was removed. The recording container was located inside a sound attenuation cabinet that contained a ventilation fan, a ceiling light, and a video camera.

Cortical EEG signals were fed via a cable attached to a commutator (Plastics-One, Roanoke, Va.), then to an amplifier (A-M Systems model 1700; 1000× gain), band pass filtered (0.3-1000 Hz), and finally digitized at 512 samples per second using ICELUS acquisition/sleep scoring software (M. Opp, U. Michigan) operating under National Instruments (Austin, Tex.) data acquisition software (Labview 5.1) and hardware (PCI-MIO-16E-4).

EEG Analysis Pre, During, and Post Seizure

EEG power (mV²/Hz) was analyzed by Fourier analysis (Fast Fourier Transform, FFT) in 1 Hz frequency bins from 1 to 96 Hz using the ICELUS software. (Although the lowest frequency bin is indicated at “0-5 Hz”, technically the lowest frequency recorded was 0.3 Hz). Frequency ranges were as follows: Delta, 0-5 Hz (0.3-4.99 Hz), Theta 5-10 Hz, Beta 10-30 Hz, Gamma—30-50 Hz, Gamma-2 50-70 Hz, Gamma-3 70-96 Hz. EEG power analysis consisted of determining the average power over successive 5 minute time periods. FFT amplitudes were log transformed to minimize biasing results by large amplitude low frequency EEG activity. The baseline EEG period from the start of recording to scopolamine administration was used to normalize EEG power across the animals, since all the animals should be in a similar activity state during this period.

To normalize the baseline, the log-FFT values for the entire baseline period and across the entire frequency range (0-96 Hz) were summed to obtain a single normalization constant Kₙₒᵣₘ:

Kₙₒᵣₘ=Σ_{f=0}⁹⁶Σₜ₌₋₁₂₀⁻¹⁰ log(FFT); f=frequency (Hz); t=time (min)

Kₙₒᵣₘ was subtracted from all FFT power values (at each frequency and all time points) for subsequent analysis. This procedure has the effect of making the average of the total baseline EEG power for each animal equal to zero, after which the baseline frequency curves for all animals should closely overlap.

EEG power data was measured beginning one hour prior to ganaxolone administration and up to one hour after administration. EEG power was averaged over 5 min periods and separated into separate wavelengths: 0-4, 4-8, 8-13, 13-30, 30-50, 50-70, and 70-96 Hz.

The present inventive concept has been described in terms of exemplary principles and embodiments, but those skilled in the art will recognize that variations may be made and equivalents substituted for what is described without departing from the scope and spirit of the disclosure as defined by the following claims.

### Example 8. Efficacy of Neurosteroid Nanoparticle Formulations in Rat Status Epilepticus Model

Seizures were induced by administering pilocarpine (50 mg/kg@5 mL/kg IP) to male Sprague-Dawley rats. 15 minutes after seizure onset, a single IV bolus dose of 3 mg/kg ganaxolone was administered intravenously either as a 30% CAPTISOL solution with a ganaxolone concentration of 2.5 mg/ml or as a 6 mg/ml ganaxolone nanoparticle suspension prepared by diluting 6 ml of the ganaxolone nanoparticle dispersion concentrate (composition shown in Table below) with 5% dextrose solution q.s to 50 ml.

Composition of ganaxolone nanoparticle dispersion concentrate

| Ingredient | wt % |
|---|---|
| Ganaxolone | 5.00% |
| Hydroxyethyl starch 130/0.4 | 10.00% |
| Sodium deoxycholate | 0.60% |
| Simethicone 30% emulsion | 0.03% |
| Deionized water | 84.37% |
| Total | 100.00% |

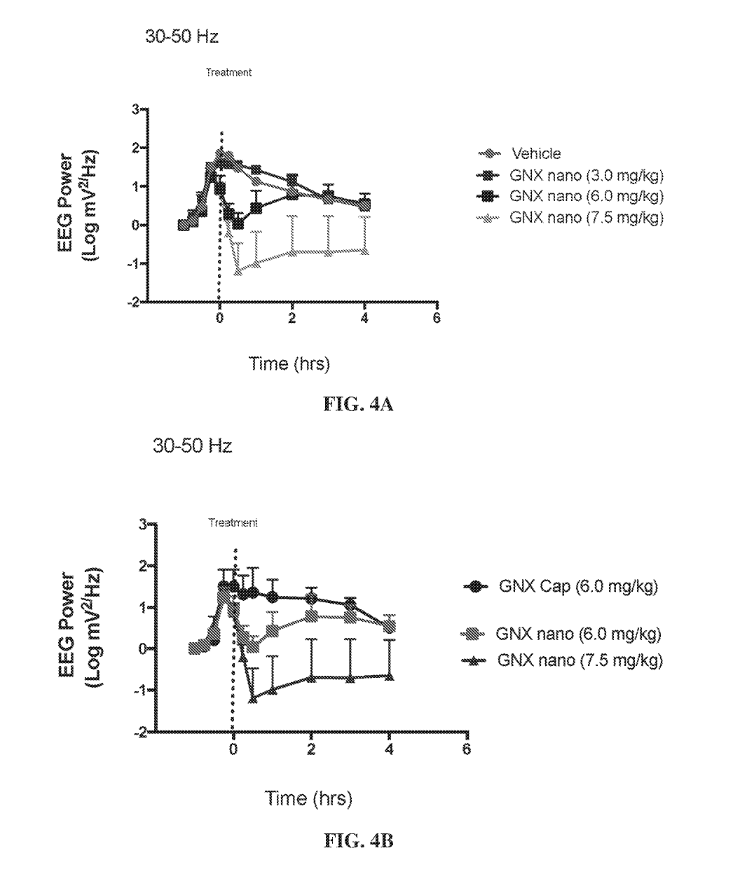

EEG Power was monitored following treatment FIG. 4A presents the dose response curve for increasing doses of the ganaxolone nanosuspension formulation. FIG. 4B presents a comparison of the ganaxolone nanosuspension and CAPTISOL formulations. This figure shows the approximately 2-fold greater potency of the nanosuspension formulation in reducing EEG power, and effecting burst suppression.

1. A method of eliciting electroencephalographic burst suppression or electroencephalographic suppression in a patient, the method comprising administering to the patient a formulation comprising neurosteroid nanoparticles having a D50 of less than 2 microns and a polymeric surface stabilizer chosen from hydroxyethyl starch, dextran, and povidone and 0.1 to 50 mg of the neurosteroid per 1 kg of the patient's body weight.

2. The method of claim 1, wherein the neurosteroid is a compound of Formula I 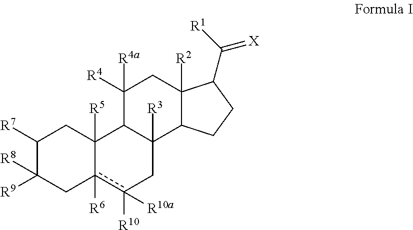
or a pharmaceutically acceptable salt thereof, wherein: [symbol]
is a double or single bond;
X is O, S, or NR¹¹;
R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;
R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆carbocycle;
R^{4a} is hydrogen or R⁴ and R^{4a} are taken together to form an oxo (═O) group;
R², R³, R⁵, and R⁶, are each independently hydrogen, hydroxyl, halogen, optionally substituted alkyl, optionally substituted cycloalkyl, or optionally substituted (cycloalkyl)alkyl, or optionally substituted heteroalkyl;
R⁷ is hydrogen, halogen, optionally substituted alkyl, optionally substituted C₃-C₆carbocycle, optionally substituted (C₃-C₆carbocycle)alkyl or —OR⁷⁰ where R⁷⁰ is hydrogen, optionally substituted alkyl, optionally substituted C₃-C₆carbocycle, or optionally substituted (C₃-C₆carbocycle)alkyl;
R⁸ is hydrogen, optionally substituted alkyl or optionally substituted C₃-C₆carbocycle, and R⁹ is hydroxyl; or
R⁸ and R⁹ are taken together to form an oxo group;
R¹⁰ is hydrogen, halogen, hydroxyl, optionally substituted alkyl, optionally substituted heteroalkyl, optionally substituted C₃-C₆carbocyle, or optionally substituted (C₃-C₆carbocycle)alkyl, and R^{10a} is hydrogen, halogen, or optionally substituted alkyl, provided that if [symbol] is a double bond R^{10a} is absent;
each alkyl is a C₁-C₁₀alkyl and optionally contains one or more single bonds replaced by a double or triple bond;
each heteroalkyl group is an alkyl group in which one or more methyl group is replaced by an independently chosen —O—, —S—, —N(R¹¹)—, —S(═O)— or —S(═O)₂—, where R¹¹ is independently chosen at each occurrence and is hydrogen, alkyl, or alkyl in which one or more methylene group is replaced by —O—, —S—, —NH, or —N-alkyl.

3. The method of claim 2 wherein the neurosteroid is ganaxolone.

4. The method of claim 2, wherein the neurosteroid is allopregnanolone, ganaxolone, alphaxalone, alphadolone, hydroxydione, minaxolone, pregnanolone, acebrochol, isopregnanolone, or tetrahydrocorticosterone or a compound of the formula

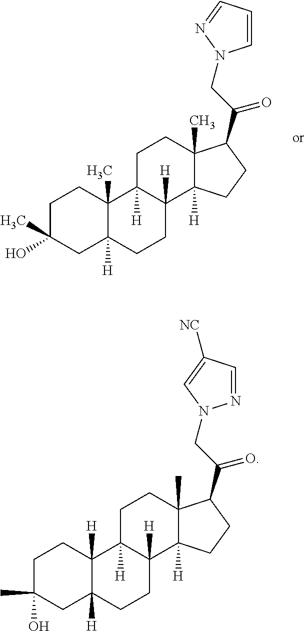

5. (canceled)

6. The method of claim 3, wherein the neurosteroid is administered as an intravenous, intramuscular, or subcutaneous injection of sequential bolus doses with an interval of less than 30 minutes between two consecutive doses.

7-9. (canceled)

10. The method of any one of claims claim 3, wherein the neurosteroid is administered intravenously as a continuous infusion.

11. The method of claim 10, wherein the neurosteroid is administered at a rate of 5 mg/hr to 300 mg/hr, 10 mg/hr to 200 mg/hr, or 20 mg/hr to 150 mg/hr.

12. The method of claim 10, wherein the neurosteroid is administered at a rate of 0.05 mg/kg/hr to 5 mg/kg/hr, 0.1 mg/kg/hr to 3.5 mg/kg/hr, or 0.2 mg/kg/hr to 2.5 mg/kg/hr.

13. The method of claim 3, wherein administration of the neurosteroid does not produce a full anesthesia effect in the patient.

14. The method of claim 3, wherein administration of the neurosteroid produces a full anesthesia effect in the patient.

15. (canceled)

16. The method of claim 3, wherein the neurosteroid formulation is an aqueous formulation comprising:
(i) nanoparticles having a D₅₀ of less than 500 nm, the nanoparticles comprising ganaxolone, wherein the weight percent of the ganaxolone is 1 to 10%;
(ii) a polymeric surface stabilizer selected from hydroxy ethyl starch, dextran, and povidone, wherein the weight percent of the polymeric surface stabilizer is 2 to 20%;
(iii) an additional surface stabilizer wherein the additional surface stabilizer is an ionic or nonionic surfactant selected sodium cholate, sodium deoxycholate, or sodium cholesterol sulfate, wherein the weight percent surfactant is 0.1% to 2.0%;
(iv) an antifoaming agent; and
the neurosteroid is ganaxolone.

17. The method of claim 1, wherein the formulation is an aqueous formulation comprising:
(i) nanoparticles having a D₅₀ of less than 500 nm, the nanoparticles comprising ganaxolone, wherein the weight percent of the ganaxolone is 5%;
(ii) a polymeric surface stabilizer selected from hydroxy ethyl starch 130/0.4 or plasdone C12, wherein the weight percent of the polymeric surface stabilizer is 10%;
(iii) an additional surface stabilizer wherein the additional surface stabilizer is sodium deoxycholate, wherein the weight percent of sodium deoxycholate is 0.75%; and
(iv) optionally simethicone, wherein the weight percent of simethicone is 0.009%.

18. A method for determining an efficacious dose of neurosteroid for treatment of an epileptic condition or effecting anesthesia, the method comprising:
intravenously, intramuscularly, or subcutaneously administering to a patient 0.1 to 50 mg of the neurosteroid per 1 kg of patient body weight;
continuously measuring an electroencephalography pattern in a brain of the patient;
detecting electroencephalographic burst suppression in the electroencephalography pattern the neurosteroid;
determining the amount of neurosteroid needed to produce the detected electroencephalographic burst suppression in the electroencephalography pattern to be the efficacious dose.

19. The method of claim 18, wherein the neurosteroid is ganaxolone, the formulation is an
injectable ganaxolone formulation comprising particles having a D₅₀ of less than 2 μm, the nanoparticles comprising
a) neurosteroid; and
b) at least one polymeric surface stabilizer selected from hydroxyethyl starch, povidone, and dextran.

20. The method of claim 19, wherein the neurosteroid is administered as sequential bolus doses with an interval of less than 30 minutes between two consecutive doses.

21. The method of claim 20, wherein the neurosteroid is ganaxolone and the ganaxolone formulation is administered every 3 minutes.

22. The method of claim 20, wherein no more than ten ganaxolone bolus doses are administered.

23. The method of claim 20, wherein dose of ganaxolone administered is at least 30 mg/kg.

24. The method of claim 20, wherein the ganaxolone is administered intravenously as a continuous infusion.

25-26. (canceled)

27. The method of claim 20, wherein administration of ganaxolone effects full anesthesia in the patient.
